(19) United States
(12) Patent Application Publication
(10) Pub. No.: US 2017/0020424 A1
(43) Pub. Date: Jan. 26, 2017

(54) Test Strip and System for Determining Measurement Data of a Probe Fluid

(71) Applicant: Infineon Technologies AG, Neubiberg (DE)

(72) Inventors: Gerald Holweg, Graz (AT); Yonsuang Arnanthigo, Villach (AT); Vijaye Kumar Rajaraman, Villach (AT)

(21) Appl. No.: 15/213,853

(22) Filed: Jul. 19, 2016

(30) Foreign Application Priority Data
Jul. 20, 2015 (DE) 10 2015 111 712.6

(51) Int. Cl. A61B 5/157; G01N 33/49; G01N 21/77; A61B 90/98; A61B 5/151; A61B 5/145; A61B 5/00; G01N 33/487; G01N 27/07

(52) U.S. Cl. CPC A61B 5/157; G01N 33/48792; G01N 33/492; G01N 21/77; G01N 27/07; A61B 5/151; A61B 5/14532; A61B 5/14539; A61B 5/14546; A61B 5/002; A61B 90/98; A61B 2562/0295

(57) ABSTRACT

A test strip comprises a test strip body comprising a fluid reservoir. The test strip further comprises a sensor unit configured to determine measurement data of a probe fluid in the fluid reservoir, and a communication unit electrically connected to the sensor unit, the communication unit including an antenna unit configured to transmit the measurement data. A system for determining measurement data of a probe fluid comprises the test strip and an external reader.

### CROSS REFERENCE TO RELATED APPLICATIONS

This application claims priority to German Application No. 10 2015 111 712.6, filed on Jul. 20, 2015, and incorporated herein by reference in its entirety.

### BACKGROUND

Blood tests and body-fluid tests are carried out on patients to determine various diseases and body condition such as glucose, salts, hormones, blood-gas, infection, viscosity, for example. Today, blood-tests are carried out in clinical laboratories or performed using point-of-care testing (POCT) at home, mainly for diabetes/glucose analysis. While the clinical procedure is manual, laborious and time-consuming, the state of the art POCT technology employs the use of three distinct devices, namely a lancet for finger-pricking, a test-strip containing electrodes and a reagent, and a dedicated electronic device for sensing/read-out of the test-strip, data display and data registration. Thus, the patients are always required to carry three distinct devices in order to successfully and safely perform the blood-test or the body-fluid tests. This may cause inconvenience to the patients, especially when travelling. It is thus desirable to provide an apparatus which facilitates point-of-care testing.

### SUMMARY

According to an embodiment of a test strip, the test strip comprises a test strip body comprising a fluid reservoir. The test strip further comprises a sensor unit configured to determine measurement data of a probe fluid in the fluid reservoir, and a communication unit electrically connected to the sensor unit, the communication unit including an antenna unit configured to transmit the measurement data.

According to another embodiment of a test strip, the test strip comprises a test strip body comprising a fluid reservoir. The test strip further comprises a sensor unit configured to determine measurement data of a probe fluid in the fluid reservoir, and a lancet fixed to the test strip body and configured to penetrate a skin of a test strip user.

According to an embodiment of a system for determining measurement data of a probe fluid, the system comprises a test strip and an external reader. The test strip comprises a test strip body comprising a fluid reservoir, a sensor unit configured to determine measurement data of the probe fluid in the fluid reservoir, and a communication unit electrically connected to the sensor unit. The communication unit includes an antenna unit configured to transmit the measurement data. The external reader is configured to transmit radio frequency energy powering the test strip and is further configured to receive the measurement data from the test strip.

Those skilled in the art will recognize additional features and advantages upon reading the following detailed description and on viewing the accompanying drawings.

### BRIEF DESCRIPTION OF THE DRAWINGS

The accompanying drawings are included to provide a further understanding of embodiments of the invention and are incorporated in and constitute a part of this specification. The drawings illustrate the embodiments of the present invention and together with the description serve to explain the principles. Other embodiments of the invention and many of the intended advantages will be readily appreciated, as they become better understood by reference to the following detailed description. The elements of the drawings are not necessarily to scale relative to each other. Like reference numbers designate corresponding similar parts.

FIG. 1 is a schematic view of a test strip according to an embodiment.

FIG. 2 is a schematic view of a test strip according to another embodiment.

FIG. 3 is schematic view of a system for determining measurement data of a probe fluid according to an embodiment.

FIG. 4A is a schematic view of a test strip comprising a lancet and a lancet cover element according to an embodiment.

FIG. 4B is a schematic view of a lancet of a test strip according to an embodiment.

FIG. 4C is a schematic view of a lancet containing a tube of a test strip according to an embodiment.

FIG. 5 is a schematic view of a test grip comprising a lancet being fixed to an end portion of a folding part being (a) accommodated in a package, being (b) in an overlapping state with the main part, and being (c) deployed such to be ready for finger-pricking.

FIGS. 6A and 6B are schematic views of a finger of a user and a test strip in a process of determining measurement data of probe fluid such as blood.

FIG. 7 is a schematic exploded view of a test strip according to an embodiment.

FIG. 8 is a schematic block diagram illustrating components of the test strip according to an embodiment.

FIG. 9 is a schematic block diagram illustrating a communication unit of the test strip according to an embodiment.

FIG. 10 is a schematic block diagram illustrating the sensor unit of the test strip according to an embodiment.

FIG. 11 is a schematic view of a sensor electrode configured to determine impedance spectroscopy data of the probe fluid according to an embodiment.

FIG. 12 is a schematic view of a sensor electrode configured to determine amperometric data of a probe fluid according to an embodiment.

FIG. 13 is a diagram illustrating a voltage applied to the sensor electrode vs. time in an impedance spectroscopy process according to an embodiment.

FIG. 14 is a diagram illustrating an amperometric current vs. a voltage applied to the sensor electrode in an amperometric measurement process according to an embodiment.

FIG. 15 is a schematic block diagram illustrating a temperature control unit of the test strip according to an embodiment.

FIG. 16 is a schematic cross-sectional view of a portion of a sensor unit, a communication unit and an energy storage unit integrated in a monolithic circuit according to an embodiment.

FIG. 17 is a schematic view of a test strip according to an embodiment.

FIGS. 18A and 18B are schematic views of a test strip according to an embodiment integrated in a cup.

### DETAILED DESCRIPTION

In the following detailed description reference is made to the accompanying drawings, which form a part hereof and in which are illustrated by way of illustration specific embodiments in which the invention may be practiced. In this regard, directional terminology such as “top”, “bottom”, “front”, “back”, “leading”, “trailing” etc. is used with reference to the orientation of the Figures being described. Since components of embodiments of the invention can be positioned in a number of different orientations, the directional terminology is used for purposes of illustration and is in no way limiting. It is to be understood that other embodiments may be utilized and structural or logical changes may be made without departing from the scope defined by the claims.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The Figures and the description illustrate relative doping concentrations by indicating “−” or “+” next to the doping type “n” or “p”. For example, “n⁻” means a doping concentration which is lower than the doping concentration of an “n”-doping region while an “n⁺”-doping region has a higher doping concentration than an “n”-doping region. Doping regions of the same relative doping concentration do not necessarily have the same absolute doping concentration. For example, two different “n”-doping regions may have the same or different absolute doping concentrations. In the Figures and the description, for the sake of a better comprehension, often the doped portions are designated as being “p” or “n”-doped. As is clearly to be understood, this designation is by no means intended to be limiting. The doping type can be arbitrary as long as the described functionality is achieved. Further, in all embodiments, the doping types can be reversed.

As employed in this specification, the terms “coupled” and/or “electrically coupled” are not meant to mean that the elements must be directly coupled together—intervening elements may be provided between the “coupled” or “electrically coupled” elements. The term “electrically connected” intends to describe a low-ohmic electric connection between the elements electrically connected together.

The present specification refers to a “first” and a “second” conductivity type of dopants, semiconductor portions are doped with. The first conductivity type may be p type and the second conductivity type may be n type or vice versa. As is generally known, depending on the doping type or the polarity of the source and drain regions, MOSFETs may be n-channel or p-channel MOSFETs. For example, in an n-channel MOSFET, the source and the drain region are doped with n-type dopants, and the current direction is from the drain region to the source region. In a p-channel MOSFET, the source and the drain region are doped with p-type dopants, and the current direction is from the source region to the drain region. As is to be clearly understood, within the context of the present specification, the doping types may be reversed. If a specific current path is described using directional language, this description is to be merely understood to indicate the path and not the polarity of the current flow, i.e. whether the transistor is a p-channel or an n-channel transistor. The Figures may include polarity-sensitive components, e.g. diodes. As is to be clearly understood, the specific arrangement of these polarity-sensitive components is given as an example and may be inverted in order to achieve the described functionality, depending whether the first conductivity type means n-type or p-type.

The terms “lateral” and “horizontal” as used in this specification intends to describe an orientation parallel to a first surface of a semiconductor substrate or semiconductor body. This can be for instance the surface of a wafer or a die.

The term “vertical” as used in this specification intends to describe an orientation which is arranged perpendicular to the first surface of the semiconductor substrate or semiconductor body.

The terms “wafer”, “substrate” or “semiconductor body” used in the following description may include any semiconductor-based structure that has a semiconductor surface. Wafer and structure are to be understood to include silicon, silicon-on-insulator (SOI), silicon-on sapphire (SOS), doped and undoped semiconductors, epitaxial layers of silicon supported by a base semiconductor foundation, and other semiconductor structures. The semiconductor need not be silicon-based. The semiconductor could as well be silicon-germanium, germanium, or gallium arsenide. According to other embodiments, silicon carbide (SiC) or gallium nitride (GaN) may form the semiconductor substrate material.

It is to be understood that the features of the various embodiments described herein may be combined with each other, unless specifically noted otherwise.

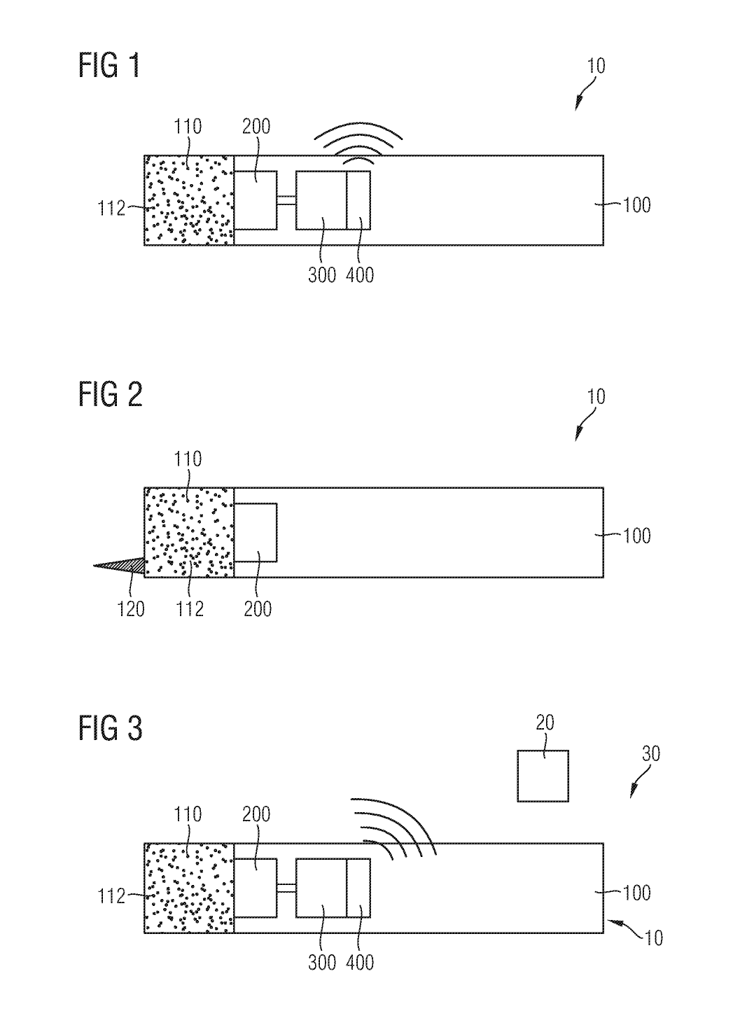

FIG. 1 is a schematic view of a test strip 10 according to an embodiment. As shown in FIG. 1, the test strip 10 comprises a test strip body 100 comprising a fluid reservoir 110. The test strip 10 further comprises a sensor unit 200, which is configured to determine measurement data of a probe fluid 112, which is received in the fluid reservoir 110. The sensor unit 200 is electrically connected to a communication unit 300. The communication unit 300 includes an antenna unit 400, which is configured to transmit the measurement data. The transmission of the measurement data by the antenna unit 400 may be wireless via radio frequency signals.

Thus, a test strip 10 is provided, which easily transmits measurement data after performing a measurement on the probe fluid 112 without the need of any further distinct device.

FIG. 2 is a schematic view of a test strip 10 according to another embodiment. As can be seen from FIG. 2, the test strip 10 comprises the test strip body 100 comprising the fluid reservoir 110. In addition, the test strip 10 of FIG. 2 comprises the sensor unit 200 configured to determine measurement data of the probe fluid 112 in the fluid reservoir 110. In addition, a lancet 120 is fixed to the test strip body 100 and configured to penetrate a skin of a test strip user.

By providing a lancet 120 fixed to the test strip body 100 of the test strip 10, no further distinct finger-pricking device is needed, thus the process of determining measurement data of the probe fluid 112 in the fluid reservoir 110 is facilitated. Furthermore, since the test strip 10 may be a disposable test strip 10 provided for single-use, the lancet 120 is also provided for single-use, thus reducing the probability of infection to a test strip user.

FIG. 3 is a schematic view of a system 30 for determining measurement data of a probe fluid according to an embodiment. The system 30 comprises the test strip 10 and an external reader 20. The test strip 10 comprises the test strip body 100 comprising the fluid reservoir 110, the sensor unit 200 configured to determine measurement data of the probe fluid 112 in the fluid reservoir 110, and the communication unit 300 electrically connected to the sensor unit 200. The communication unit 300 includes an antenna unit 400 configured to transmit the measurement data, for example via radio frequency signals. The external reader 20 is configured to transmit radio frequency energy powering the test strip 10 plus optionally to transmit radio frequency data to the test strip 10 and is further configured to receive the measurement data from the test strip 10, e.g. by receiving radio frequency signals from the test strip 10 related to the measurement data.

By providing the system 30 for determining measurement data of a probe fluid 112, a new approach for point-of-care testing (POCT) of blood, of a body-fluid or any fluid to be tested is provided by using a so-called smart test strip having an integrated sensing and data transmission capability to an external reader 20 such as a cellular phone, a personal computer, a tablet personal computer or a watch. The electric components of the test strip 10 such as the sensor unit 200, the communication unit 300 and the antenna unit 400 may be integrated directly into the test strip. Thus, a patient is allowed to measure at lest one blood-related parameter or body-fluid related parameter at their homes using a POCT device, instead of clinical/laboratory-based testing.

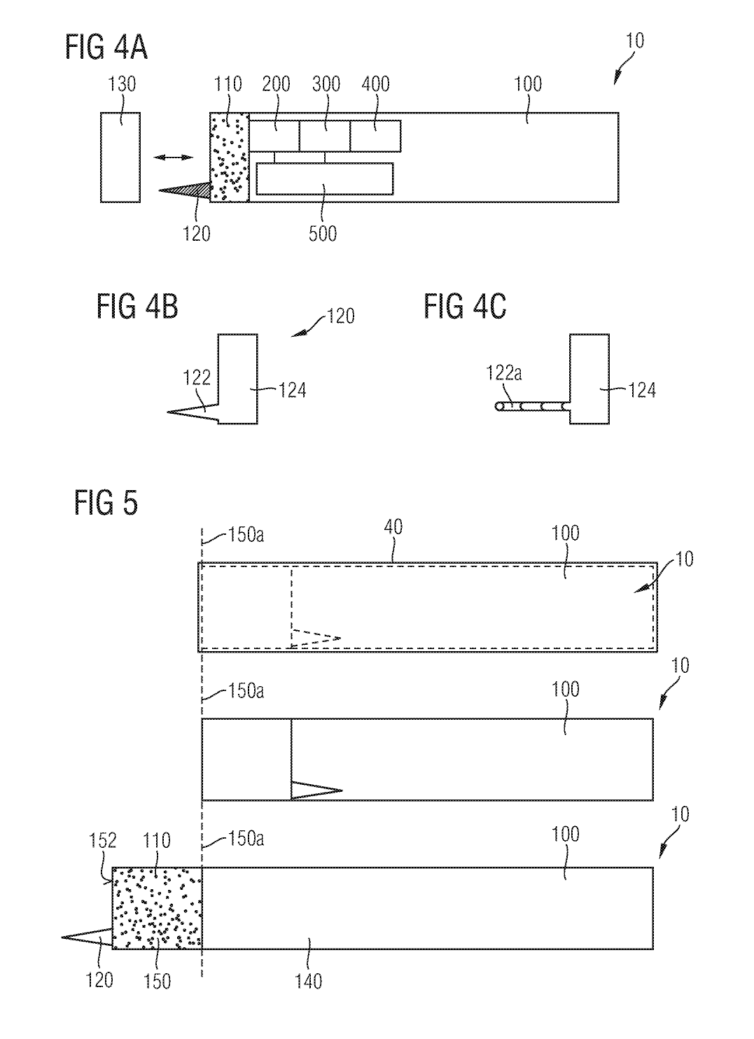

FIG. 4A is a schematic view of a test strip 10 according to an embodiment. As can be seen from FIG. 4A, the test strip 10 comprises the test strip body 100. At an end portion of the test strip body 100, the lancet 120 is fixed. The lancet 120 is configured to penetrate a skin of a test strip user, in order to take a blood sample to be used as the probe fluid 112 in a blood sensing process of the test strip 10. The lancet 120 may comprise a metal such as a stainless steel or any metallic alloy being adapted to be stored without any or with reduced rust and oxidation.

As shown in FIG. 4B, the lancet 120 may comprise a lancet part 122 and a base part 124. The lancet 120 is fixed to the end portion of the test strip body with the base part 124 such that the lancet part 122 protrudes from the end portion of the test strip body 100. The lancet part 122 and the base part 124 may be integrally formed from one piece of metal, e.g. by stamping or by cutting. The lancet 120 may be formed from a metal sheet or a steel blade. The lancet part 122 of the lancet 120 may have a triangular sheet form, wherein the protruding edge of the triangular lancet part 122 has an sharp angle configured to penetrate a skin of a test strip user. The lancet part 122 may also have a needle-shape.

For storing and transport purposes of the test strip 10, a lancet cover element 130 may be provided, which is configured to accommodate the lancet 120. The lancet cover element 130 may comprise a synthetic material. The test strip body 100 may also comprise a synthetic material. The base part 124 of the lancet 120 may be fixed to the test strip body 100 by gluing. In addition, the lancet 120 may be form-locked within the test strip body 100 by its base part 124, wherein the lancet part 122 protrudes from the test strip body 100. The lancet 120 may be fixed at an end portion of the test strip body 100, where the fluid reservoir 110 is provided.

As can be seen from FIG. 4C, the lancet 120 may comprise a tube 122a mounted on the base part 124. The tube 122a may be capable to transport the probe fluid 112 (such as water or blood) by capillary effect directly from its distal end at a skin penetrating peak to the fluid reservoir 110. The lancet 120 thus may comprise the tube 122a capable to transport the probe fluid 112 by capillary effect to the fluid reservoir 110.

The fluid reservoir 110 is a portion of the test strip body 100 being adapted to receive a fluid such as a body fluid, blood, urine, of a human or an animal, for example. The fluid may, however, also be a fluid to be probed in an environmental investigation. Thus, the probe fluid 112 may also be water of a lake or a river, the quality of which has to be tested. In addition, the probe fluid 112 may also be water contained in a swimming pool. In such a case, the lancet 120 may also be omitted. In the test strip body 100, the sensor unit 200, the communication unit 300 and the antenna unit 400 are integrated. The test strip 10 may further comprise an energy storage unit 500 electrically connected to the sensor unit 200 and the communication unit 300, to supply electric energy to the sensor unit 200 and the communication unit 300.

The fluid reservoir 110 may comprise a porous material adapted to absorb the probe fluid 112 and further to contain the probe fluid 112 to be tested by the sensor unit 200. The sensor unit 200 comprises a sensor being in contact with the fluid reservoir 110 and, in a measurement process, being in contact with the probe fluid 112 contained in the fluid reservoir 110. Thus, by providing the fluid reservoir 110, the probe fluid 112 may be soaked, sponged up or sucked up by the fluid reservoir 110 comprising a porous material. The fluid reservoir 110 and the test strip body 100 may comprise the same material such as a synthetic material, wherein the material within the fluid reservoir 110 is made porous. It is also possible to provide the fluid reservoir 110 as a distinct element comprising, for example, an absorbent paper.

Although the test strip 10 in FIG. 4A is shown to include an antenna unit 400, it is also possible to provide the sensor unit 200 and the communication unit 300 only, wherein the communication unit 300 is adapted to communicate with an external reader by inserting a communication plug into a built-in slot of the external reader 20 such as a smart-phone, a tablet PC, a smart watch, a laptop or a personal computer. According to an embodiment, the test strip 10 of FIG. 4A may also comprise the test strip body 100 comprising the fluid reservoir 110 and the lancet 120 only. In such an embodiment, the analysis of the probe fluid 112 may be done by a distinct analysis apparatus, e.g. by an optical analysis of a reagent contained in the fluid reservoir 110 of the test strip body 100. Furthermore, an electrical interface may be provided at the test strip 10 of FIG. 4A in which sensor electrodes of the sensor unit 200 are directly connected without any processing units interconnected to an analysis apparatus in the external reader 20.

Thus, the test strip 10 may include a lancet 120 for finger-pricking, wherein, in a first approach, the test strip 10 with on-strip analysis is inserted into a built-in slot in a smart-phone or a tablet PC, or a smart watch, or a laptop, or a personal computer to read out measured values, display and record test results. In a second approach, the test strip 10 with on-strip analysis may directly transmit measured values to the external reader 20. Here, an energy storage unit 500 like a MEMS battery or an energy harvester may be included in the test strip 10. In a third approach, a semi-smart test strip may be provided, which contains only electrodes and is inserted into a built-in slot in an external reader 20 such as a smart-phone, a tablet PC, a smart watch, a laptop or a personal computer, The external reader 20 may contain the sensor unit 200 to analyze, display and record test results. Thus, a smart or a semi-smart test strip for point-of-care-testing is provided, which has an integrated lancet 120 and eliminates the requirement of intermediate dedicated electronic device read-out/display device. The external reader 20 may be used for either just displaying and registering the blood-parameter values from the test strip 10 (first or second approach) or sensing, displaying and registering the blood parameter value when using a semi-smart test strip (third approach). The above concepts use a blood sensor capable of impedance spectroscopy or amperometric sensing, for example. The second approach employs direct wireless communication through an integrated energy storage unit, while the first and third approach employs a slot-based approach, where energy is provided by the external reader 20.

FIG. 5 shows a test strip 10 according to another embodiment. As can be seen from FIG. 5, the test strip body 100 comprises a main part 140 and a folding part 150. The lancet 120 is fixed to an end portion of the folding part 150 such that the lancet 120 is overlapping the main part 140 in a folded state of the folding part 150, and such that the lancet 120 is protruding from the folding part 150 in an unfolded state of the folding part 150. In a storing or transport state, the test strip 10, which may be a disposable test strip 10, is accommodated in a package 40. The package 40 may be an aseptic package 40 ensuring an aseptic state of the test strip 10 and the lancet 120. The package 40 is configured to accommodate the test strip body 100 having the folding part 150 in a folded state. The folding part 150 may be folded or bend in relation to the main part 140 along a folding line 150a. When using the test strip 10 of FIG. 5, the package 40 is opened by a user by ripping the package 40. The package 40 may comprise a synthetic material and/or a paper material. The package 40 may also comprise a paper material, which is covered by a synthetic material or a metal material at the inside of the package 40. After opening the package 40, the test strip 10 is taken out from the package 40, as can be seen from FIG. 5(b). Thereafter, as can be seen from FIG. 5(c), the folding part 150 is unfolded such that the lancet 120 is freely exposed and not overlapping the main part 140 anymore.

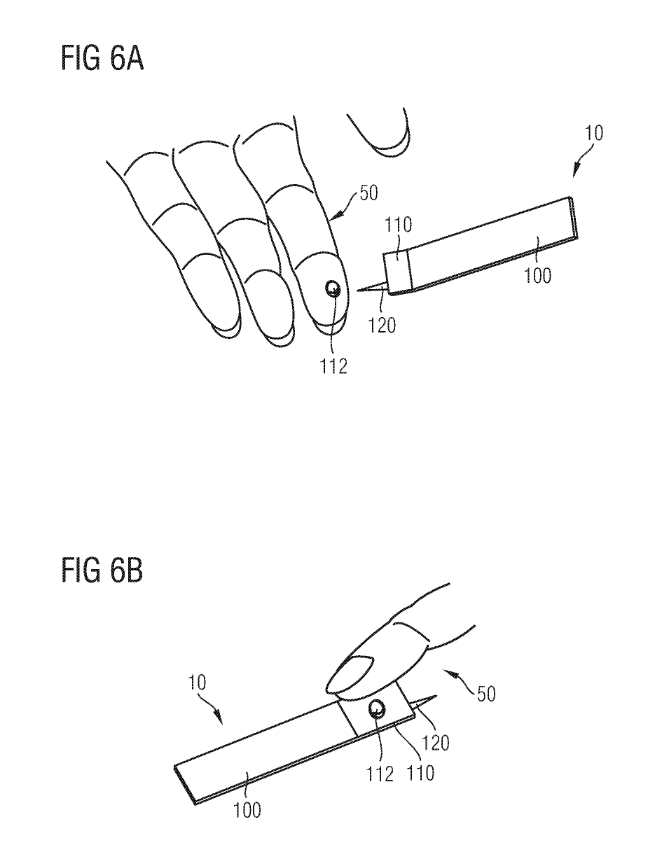

FIGS. 6A and 6B show a measurement process of a test strip 10 for determining measurement data of a probe fluid 112 received in a fluid reservoir 110 of a test strip 10. As can be seen from FIG. 6A, the lancet 120 is used for finger-pricking of a finger 50 of a test strip user. By penetrating the skin of the finger 50, a blood sample constituting the probe fluid 112 can be taken from the test strip user. As can be seen from FIG. 6B, the test strip user presses the finger 50 on the fluid reservoir 110 to bring the probe fluid 112 in contact with the fluid reservoir 110, in which the probe fluid 112 is absorbed. As already discussed above, measurement data of the probe fluid 112 are then generated by the sensor unit 200, which has a sensor being in contact with the fluid reservoir 110 and the probe fluid 112.

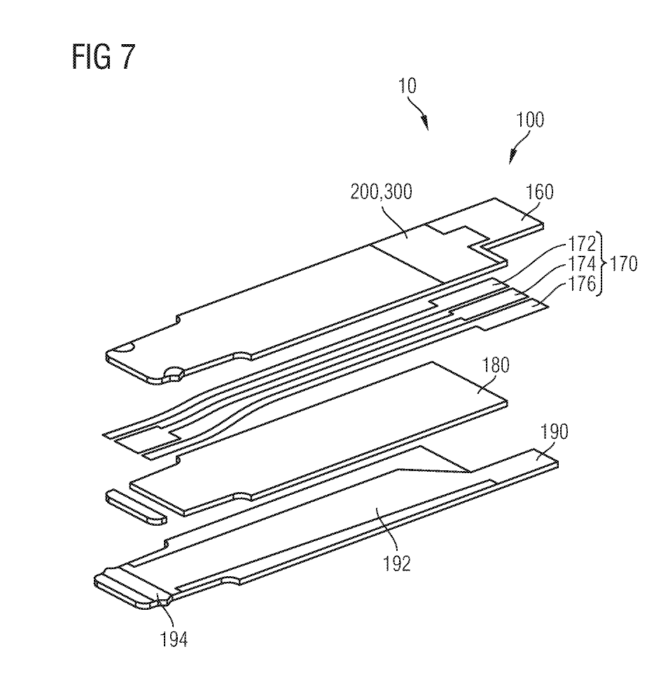

FIG. 7 is an exploded schematic view of a test strip 10 according to an embodiment. As can be seen from FIG. 7, the test strip 10 may be assembled in a layer structure. Herein, the test strip body 100 may comprise a top synthetic layer 160, in which the electronic components such as the sensor unit 200 and the communication unit are integrated. On a backside of the top synthetic layer 160, an electrode layer 170 may be provided, which contains a first indicator electrode 172, a counter reference electrode 174 and a second indicator electrode 176. The counter reference electrode may comprise an Ag/AgO-material. On a backside of the electrode layer 170, an adhesive layer 180 may be provided. On the backside of the adhesive layer 180, a carbon working electrode 192 formed on a bottom synthetic layer 190 may be formed. The bottom synthetic layer 190 forms the bottom part of the test strip body 100, wherein the top synthetic layer 160 forms the top part of the test strip body 100. On the carbon working electrode 192, a reagent 194 may be provided for determining measurement data of the probe fluid 112.

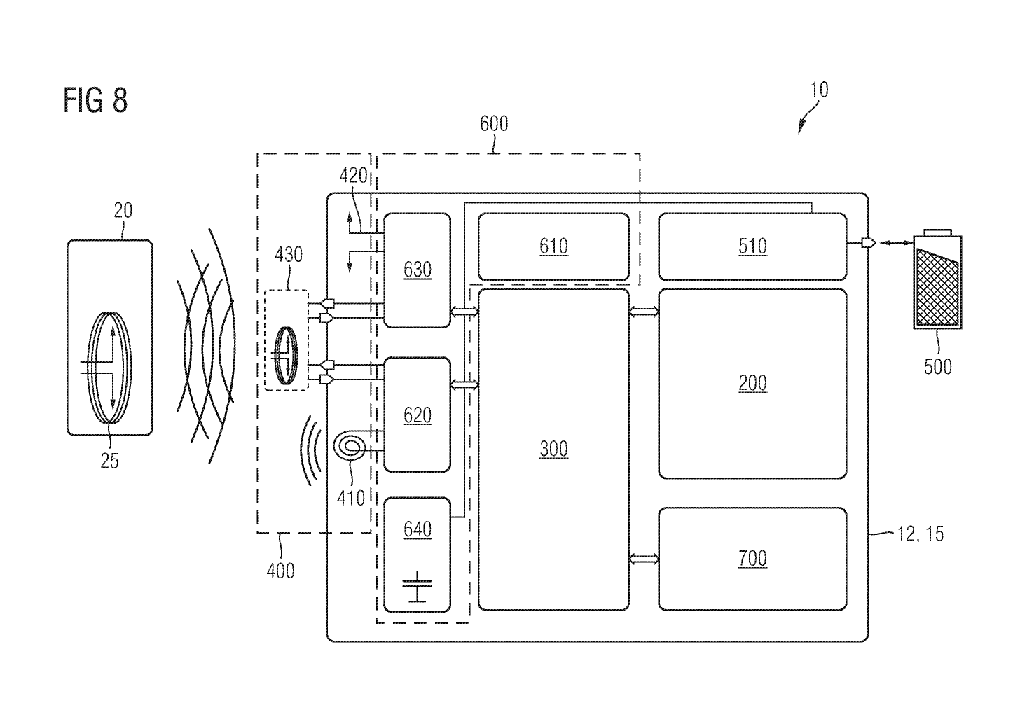

FIG. 8 is a schematic block diagram of electronic components integrated in a test strip 10 according to an embodiment. As can be seen from FIG. 8, the test strip 10 comprises the sensor unit 200, the communication unit 300 and the antenna unit 400. Further to the sensor unit 200, the communication unit 300 and the antenna unit 400, the test strip 10 may comprise an energy storage unit 500 connected to the sensor unit 200 and the communication unit 300.

The energy storage unit 500 may comprise a DC/DC-converter 510 for converting between the voltage supplied by the energy storage unit 500 and an operating voltage of the electronic components of test strip 10. The energy storage unit 500 may comprise a chargeable storage device. Herein, the chargeable storage device may comprise a silicon-based rechargeable lithium battery. As silicon has highest lithium ion storage capacity/volume, even a battery having a size lower than 1 mm² may provide a storage capacity in the order to up to 250 to 500 μAh. The silicon-based rechargeable lithium battery may have a size in a range between 1 mm² to 20 mm². The silicon-based rechargeable lithium battery may have an energy storage capacity in a range between 0.01 mAh to 2 mAh and an operating voltage in a range between 2 V to 5 V. The energy storage unit 500 may further comprise a capacitor. Herein, printed or silicon integrated energy storage devices or supercapacitors may be used. The capacitor may have a size in a range between 1 mm² to 15 mm² and may have a capacitance of 0.5 μF to 20 μF at a voltage of 1.5 V to 15 V.

The antenna unit 400 may comprise at least one of a radio frequency identification (RFID)/nearfield communication (NFC) antenna 410 and a radio frequency identification (RFID)/ultra-high frequency (UHF) antenna 420. The antennas 410 and 420 may be integrated in a monolithic circuit 12 together with the sensor unit 200 and the communication unit 300. Optionally, external antennas 430 adapted for high frequency (HF) and/or ultra-high frequency (UHF) radio frequency identification (RFID) communication may be provided.

RFID devices operate at different radio frequency ranges, e.g. low frequency (LF) at about 28 to 135 kHz, high frequency (HF) at about 13.56 MHz, and ultra-high frequency (UHF) at 860 to 960 MHz. Each frequency range has unique characteristic in terms of RFID performance.

NFC is a short range technology that enables two devices to communicate when they are brought into actual touching distance. NFC enables sharing power and data using magnetic field induction at 13.56 MHz (HF) band, at short range, supporting varying data rates from 106 kbps, 212 kbps to 424 kbps. A key feature of NFC is that is allows two devices to interconnect. In reader/writer mode, an NFC tag is a passive device that stores data that can be read by an NFC enable device. In peer-to-peer mode, two NFC devices can exchange data. Bluetooth or WiFi link set up parameters can be shared using NFC and data such as virtual business cards or digital photos can be exchanged. In card emulation mode, the NFC device itself acts as an NFC tag, appearing to an external interrogator as a traditional contact less smart card. These NFC standards are acknowledged by major standardisation bodies and based on ISO/IEC 18092.

Passive UHF systems use propagation coupling, where an interrogator antenna emits electromagnetic energy radio frequency waves and the RFID tag receives the energy from the interrogator antenna, and the integrated circuit uses the energy to change the load on the antenna and reflect back an altered signal that is then demodulated. For the LF and HF RFID systems using interactive coupling, the range of the interrogator field is small (0.2 to 80 cm) and can be relatively easily controlled. UHF systems that use propagation coupling are harder to control, because energy is sent over long distances. The radio waves can reflect on hard surfaces and reach tags that are not in the normal range. LF and HF systems perform better than UHF systems around metal and water. The radio waves do reflect off metal and cause false reads, and they are better able to penetrate water. UHF radio waves are attenuated by water.

In addition, communication may be performed via any one of an Industrial, Scientific and Medical (ISM) Band, which operates in a frequency range between 6.765 MHz to 246 GHz and has bandwidths of up to 2 GHz.

The test strip 10 may further comprise an energy harvesting unit 600 configured to harvest energy from an external power source, wherein the energy harvesting unit 600 is connected to the antenna unit 400. The energy harvesting unit 600 may comprise a power management unit 610, a high frequency (HF) power converter unit 620 being connected to the radio frequency identification (RFID)/nearfield communication (NFC) antenna 410, and an ultra-high frequency (UHF) power converter unit 630 being connected to the radio frequency identification (RFID)/ultra-high frequency (UHF) antenna 420.

Energy may be harvested through a dedicated radio frequency source such as the external reader 20 comprising an antenna unit 25 being adapted for RFID/NFC communication and/or RFID/UHF communication. The energy may also be harvested from ambient radio frequency. The HF power converter unit 620 connected to the RFID/NFC antenna 410 is able to harvest energy from different external readers 20 such as smart phones or RFID readers to power data transmission. The UHF power converter unit 630 connected to the RFID/UHF antenna 420 is able to harvest ambient radio frequency energy from existing external energy sources like TV signal, WiFi/WiMAX, GSM an others.

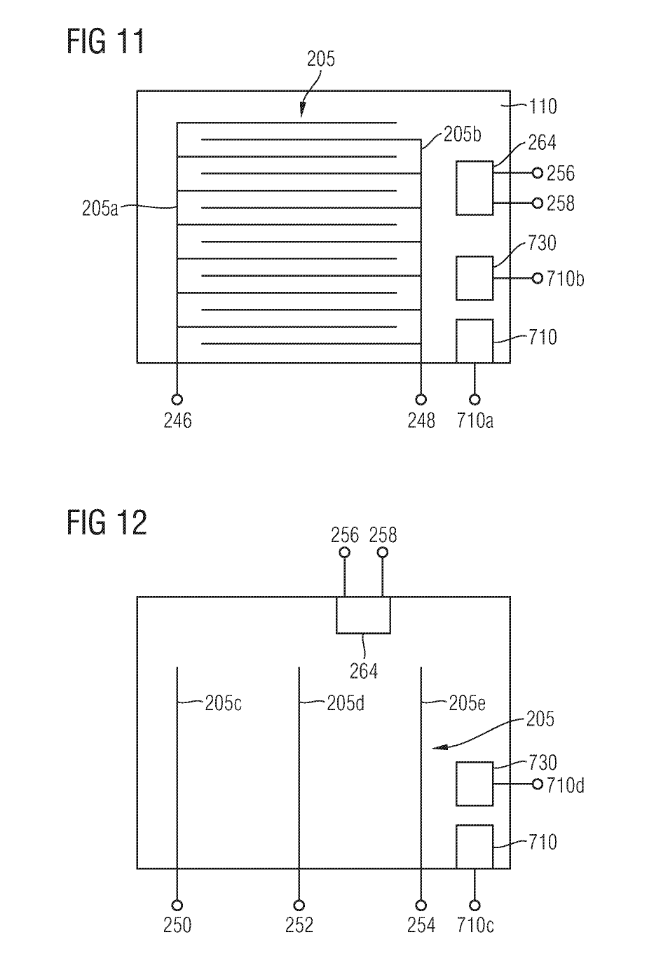

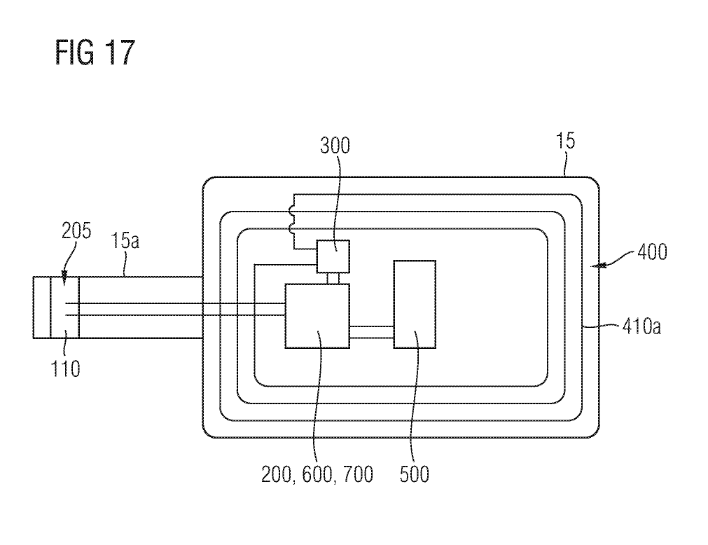

Furthermore, the energy harvesting unit 600 may comprise a capacitor 640 for storing electric energy to be provided to the energy storage unit 500. In addition, the test strip 10 may further comprise a temperature control unit 700, which is configured to regulate the temperature of the fluid reservoir 110 in the test strip body 100 of the test strip 10. A temperature sensor 710 (FIGS. 11 and 12) may be provided, which is configured to determine the temperature of the fluid reservoir 110. The electronic components as described above, i.e. the sensor unit 200, the communication unit 300, the antenna unit 400, the energy storage unit 500, the energy harvesting unit 600 and the temperature control unit 700 may be integrated in a monolithic circuit 12, which is embedded in the test strip body 100 of the test strip 10. However, at least one of the electronic components 200 to 700 may also be omitted in the monolithic circuit 12 and provided as an external circuit embedded in the test strip body 100. Furthermore, at least one of the electronic components as described above, i.e. the sensor unit 200, the communication unit 300, the antenna unit 400, the energy storage unit 500, the energy harvesting unit 600 and the temperature control unit 700 may be mounted on a printed circuit board 15, which is embedded in the test strip body 100 of the test strip 10, as shown in FIG. 17.

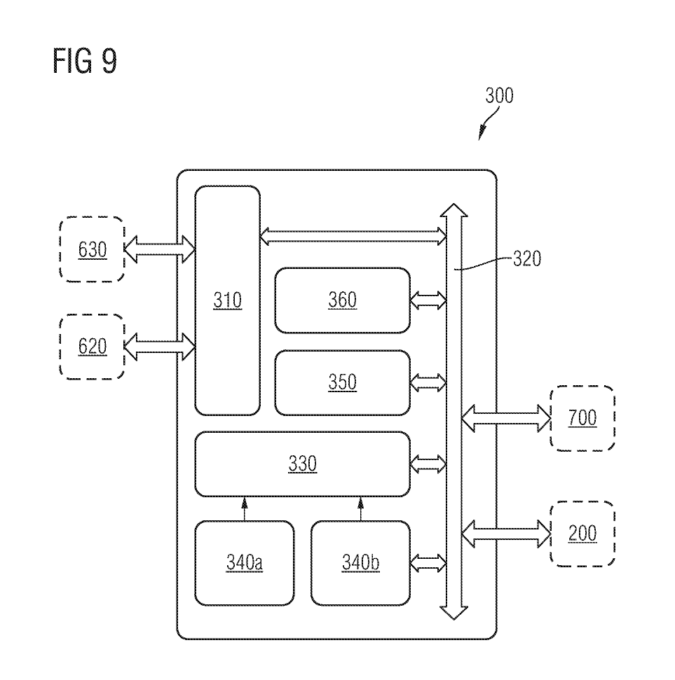

FIG. 9 is a schematic block diagram of the communication unit 300. As can be seen from FIG. 9, the communication unit 300 comprises an High frequency/ultra high frequency radio frequency (HF/UHF RF) digital front end unit 310. The HF/UHF RF digital front end unit 310 is connected to the RFID/NFC antenna 410 via the HF power converter unit 620, and is further connected to the RFID/UHF antenna 420 via the UHF power converter unit 630. The HF/UHF RF digital front end unit 310 may be accessed via a standard RFID reader such as the external reader 20 or an nearfield communication capable cell phone constituting the external reader 20. The RFID communication may be performed in a frequency range between 10 MHz to 20 MHz, or at 13.56 MHz, which is a standard RFID communication radio frequency.

The HF/UHF RF digital front end unit 310 may also communicate via the RFID/UHF antenna 420 by means of an UHF/RFID interface. The radio communication frequency for UHF/RFID communication may be in a range between 800 to 900 MHz, or at 868 MHz.

The HF/UHF RF digital front end unit 310 may communicate with the sensor unit 200 or the temperature control unit 700 via write-read commands transmitted on a system bus 320, as indicated in FIG. 9.

A microcontroller 330 is provided in the communication unit 300, which is adapted to handle an radio frequency protocol. The microcontroller 330 may be electrically connected to a read-only memory 340a for storing RFID firmware and/or to a pseudo read-only memory 340b for storing prototyping firmware. In addition, a random access memory 350 may be connected to the system bus 320 for buffering measurement raw data of the sensor unit 200 or processed measurement data determined by an analysis of the measurement raw data. Furthermore, a timer unit 360 may be provided and electrically connected to the system bus 320 for providing the communication unit 300 with a clock.

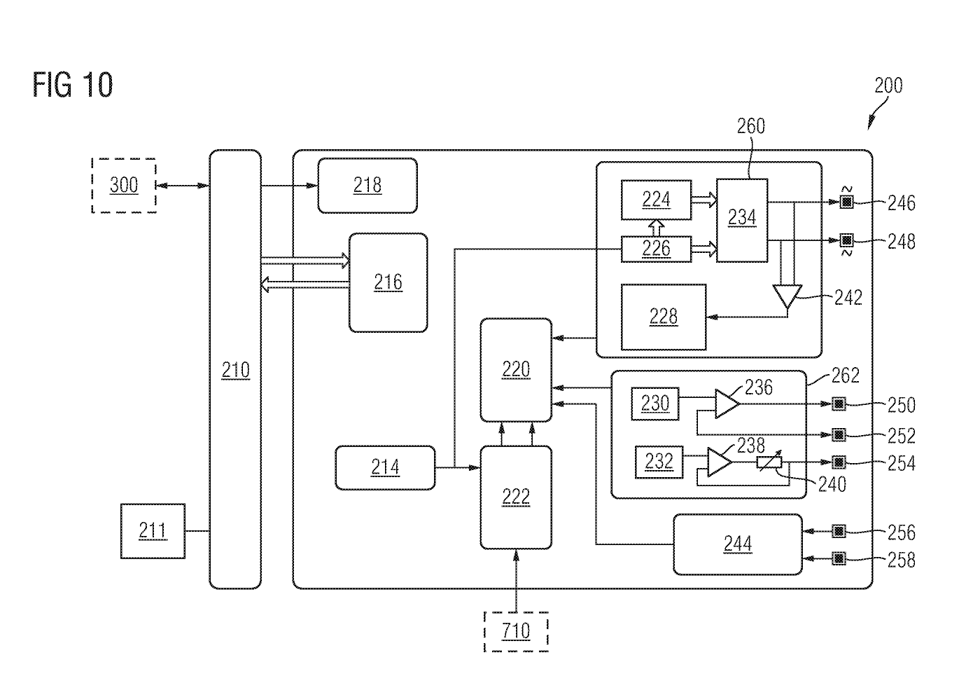

FIG. 10 is a schematic block diagram of the sensor unit 200 of the test strip 10 according to an embodiment. As can be seen from FIG. 10, the sensor unit 200 comprises a sensor bus 210, which may be connected to the system bus 320 of the communication unit 300. The sensor unit 200 further comprises a voltage regulator unit 218, which is connected to the sensor bus 210. The sensor bus 210 may be connected to a data management unit 211, which is adapted to process and manage the measurement data determined by the sensor unit 200 of the probe fluid 112. To achieve very high precision and sensitivity levels at the sensor interface, a separate voltage regulator unit 218 is implemented to provide a constant and stable supply voltage for the sensor unit 200.

The sensor bus 210 is further connected to a control logic unit 216, which is adapted to control the measurement processes performed by the sensor unit 200. A reference generator unit 222 is provided with temperature data of the fluid reservoir 110 measured by the temperature sensor 710, which will be discussed in detail below. The reference generator unit 222 is further provided with a clock rate by an oscillator unit 214. The reference generator unit 222 is connected to an analog digital converter 220, which converts the analog measurement data into digital measurement data to be provided to the sensor bus 210 and the data management unit 211.

The sensor unit 200 may comprise a sensor electrode unit 205 (FIGS. 11 and 12) which is configured to determine amperometric data or impedance spectroscopy data of the probe fluid 112 received in the fluid reservoir 110 of the test strip 10. The impedance spectroscopy data may be determined by an impedance spectroscopy unit 260. The amperometric data may be determined by the amperometric measurement unit 262. Furthermore, an interface unit 244 may be provided in the sensor unit 200 for connecting additional sensors. According to an embodiment, the sensor unit 200 may comprise an optical sensor 264, which is configured to determine optical data of the probe fluid 112. The optical sensor 264 may be connected to the interface unit 244 via the connecting terminals 256, 258.

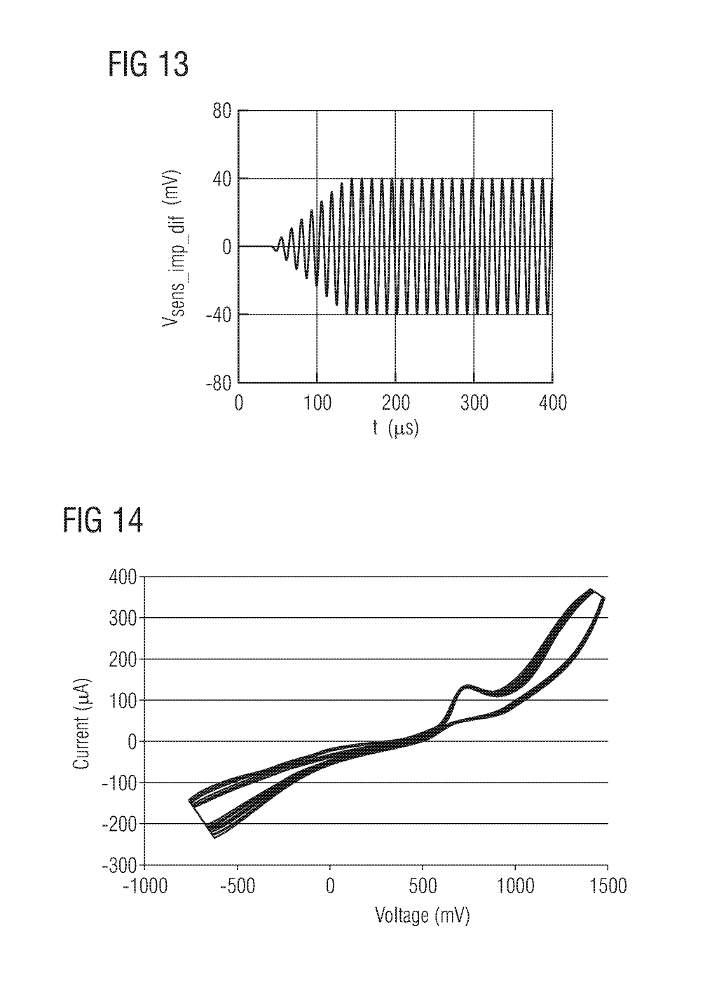

The impedance spectroscopy unit 260 comprises a ramp generation unit 226, which receives a clock signal from the oscillator unit 214. The ramp signal from the ramp generation unit 226 is transmitted to a sine lookup table unit 224 and to a current steering digital analog conversion unit 234. The sine signal of the sine lookup table unit 224 is transmitted also to the current steering digital analog converting unit 234. The impedance spectroscopy signal output to the sensor electrode unit 205 is shown in FIG. 13. According to an embodiment, the sensor electrode unit 205 may comprise interdigitated electrodes 205a, 205b having respective connection terminals 246, 248, of the sensor unit 200. As can be seen from the output signal in FIG. 13, the sensor electrode unit 205 as shown in FIG. 11 may be excited with a sinusoidal current in the range of 1 μA up to 1 mA and a frequency of 100 Hz up to 2 MHz. The resulting voltage between the interdigitated electrodes 205a, 205b and thus between the connection terminals 246 and 248 is then amplified by an amplifier unit 242 and transmitted to an amplitude and phase detector unit 228 of the impedance spectroscopy unit 260. The resulting measurement data of the impedance spectroscopy unit 260 is transmitted to the analog digital converter unit 220, which in turn, transmits the digital measurement data to the sensor bus 210. The digital measurement data may be further processed in the data management unit 211. The digital measurement data is then transmitted to the communication unit 300 to be transmitted to the external reader 20.

As can be further seen from FIG. 11, the temperature sensor 710 and the optical sensor 264 may be provided in the fluid reservoir 110, to measure a temperature or optical data of the probe fluid 112 received in the fluid reservoir 110. The optical data from the optical sensor 264 may be transmitted from the interface unit 244 to the analog digital converting unit 220, which is then transmitted to the sensor bus 210 for further transmission by the communication unit 300.

Furthermore, analog measurement data of the amperometric measurement unit 262 may be transmitted to the analog digital converting unit 220 and then transmitted to the sensor bus 210 and the communication unit 300.

The amperometric measurement unit 262 is connected to the sensor electrode unit 205 having sensor electrodes as shown in FIG. 12, for example. As can be seen from the embodiment of FIG. 12, the sensor electrode unit 205 comprises three sensor electrodes, i.e. a working electrode 205c connected to a connection terminal 250, a reference electrode 205d connected to a connection terminal 252 and an auxiliary electrode 205e connected to a connection terminal 254. Optionally, the temperature sensor 710 and the optical sensor 264 may be provided in the fluid reservoir 110 of the test strip 10 as shown in FIG. 12. The working electrode 205c, the reference electrode 205d and the auxiliary electrode 205e are connected to respective connection terminals 250, 252 and 254 of the amperometric measurement unit 262 in the sensor unit 200. The working electrode 205c is provided with a constant current generated by an digital analog converting unit 232, an operation amplifier 238 and a variable resistance 240. The auxiliary electrode 205e is connected to an output of an operation amplifier 236 having its first input connected to an analog digital converter 230 and its second input connected to the reference electrode 205d.

An example of a characteristic curve of the amperometric data of the amperometric measurement unit 262 is shown in FIG. 14. A voltage range across the sensor electrodes is in a range between +/−5 V or +/−3 V or +/−2 V. A drive current may be in a range up to 500 μA. The measured currents are in a range between 100 nA and 1 μA.

By providing the amperometric measurement unit 262, a differential measurement may be performed by using a reference spectrum. A reference spectrum may be determined from a measurement process of water having a conductance in a range between 300 to 800 μS at a temperature of 20° C.

By measuring a diffusion threshold current between the polarisable auxiliary and working electrode and the reference electrode at a constant potential, a salt concentration can be derived when the temperature and the potential are known. Thus, a salt concentration of the probe fluid 112 may be determined.

By the optical sensor 264, the sensor electrode unit 205 of the impedance spectroscopy unit 260, and by the sensor electrode unit 205 of the amperometric measurement unit 262, measurement data of the probe fluid 112 may be determined, which is indicative of one of a glucose concentration, a ph-value, a salt concentration, a potassium concentration, a concentration of a chemical substance, a concentration of a biochemical substance, or a conductivity value of the probe fluid 112 in the fluid reservoir 110. The test strip 10 is thus adapted to measure a multitude of different fluid parameters of different fluids. The selectivity of the sensor electrode unit 205 may be achieved by a respective functionalisation of the electrode surfaces of the sensor electrode unit 205. It should be emphasized that the sensor unit 200 may comprise a multitude of sensors or sensor electrode units 205 each selectively functionalized to measure a respective fluid parameter. Furthermore, a multitude of sensor units 200 may be integrated in the test strip 10, each being adapted to measure respective measurement data by means of respective functionalised sensor electrode units 205.

The measurement data, which is converted from an analog to a digital form in the analog digital conversion unit 220 may be further processed by the data management unit 211 connected to the sensor bus 210, which is, in turn connected to the analog digital conversion unit 220. By processing of the digital measurement data, the data management unit 211 may determine a fluid parameter of the probe fluid 112 such as the glucose concentration, the pH-value the salt concentration or the conductivity value. Thus, only a fluid parameter may be transmitted to the communication unit 300, reducing the amount of data to be transmitted to the external reader 20.

For processing the measurement data, external configuration data may be necessary to determine a fluid parameter of the probe fluid 112. According to an embodiment, the external configuration data may be related to body related data of a test strip user. The external configuration data may be transmitted from the external reader 20 to the communication unit 300, to be used for processing the digital measurement data transmitted from the analog digital conversion unit 220 to the sensor bus 210. Thus, according to an embodiment, the antenna unit 400 may be further configured to receive external configuration data.

According to an embodiment, the antenna unit 400 may constitute the sensor electrode unit 205. Thus, according to an embodiment, the antenna unit 400 may comprise the interdigitated electrodes 205a, 205b having the connection terminal 246 and the connection terminal 248, respectively, to transmit the measurement data to the external reader 20 via radio frequency signals in a ultra-high frequency range or a high frequency range. According to another embodiment, the antenna unit 400 may comprise at least two of the sensor electrodes 205c, 205d, and 205e of the sensor electrode unit 205 as shown in FIG. 12. In order to prevent an interference of the electronic components of the impedance spectroscopy unit 260 or the amperometric measurement unit 262, a switching unit may be interconnected between the antenna unit 400 and the sensor unit 200, to switch the connection terminals 246 and 248 or the connection terminals 250 to 254 between a connection with the sensor unit 200 and the antenna unit 400.

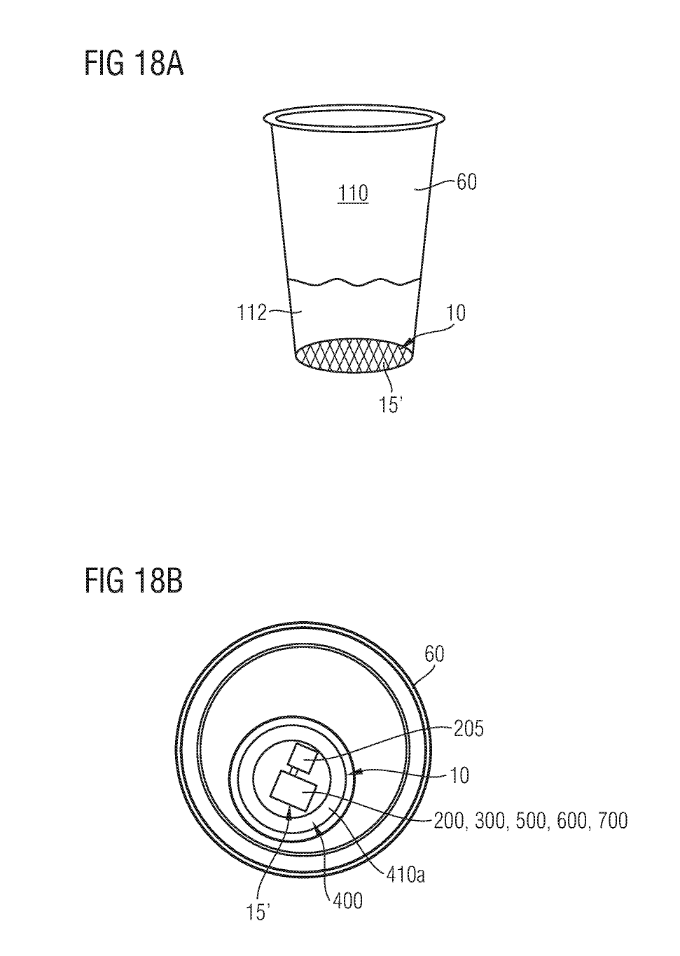

In case the antenna unit 400 constitutes a sensor electrode unit 205, the area consumption of the fluid sensing system comprising the sensor unit 200, the communication unit 300 and the antenna unit together with the sensor electrode unit 205 may be reduced. According to another embodiment, the antenna unit 400 may comprise an inductive coil antenna surrounding the sensor electrode unit 205. According to still another embodiment as shown in FIGS. 17 and 18B, an inductive coil antenna 410a may be arranged on a printed circuit board (PCB) surrounding the sensor unit 200, the communication unit 300, the energy storage unit 500, the energy harvesting unit 600 and the temperature control unit 700 mounted on the PCB.

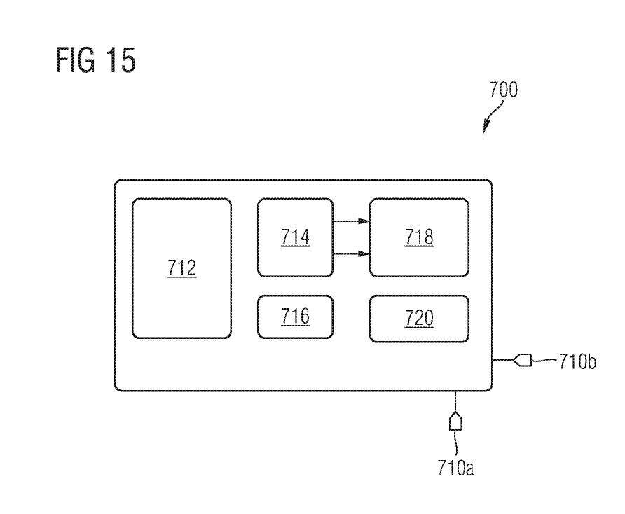

FIG. 15 is a schematic block diagram of a temperature control unit 700 of a test strip 10 according to an embodiment. The temperature control unit 700 comprises a configuration control and result unit 712, a low noise bandgap unit 714, a timer unit 716, a successive approximation register (SAR) analog-to-digital converter (ADC) unit 718 and a minimum/maximum-comparator unit 720. The temperature control unit 700 is adapted to measure on-chip supply voltages and battery voltage during a normal operation. The temperature control unit 700 can further measure the chip temperature. By providing the temperature control unit 700, ultra-low current consumption in a polling mode is available.

The temperature control unit 700 is connected to the temperature sensor 710 via the connection terminal 710a. The temperature control unit 700 may further comprise a heating device 730 (FIG. 11 and FIG. 12), which is connected to the temperature control unit 700 via a connection terminal 710b. Thus, the temperature control unit 700 is adapted to measure the temperature of the probe fluid 112 and the fluid reservoir 110 by means of the temperature sensor 710, and to further regulate the temperature in the probe fluid 112 and the fluid reservoir 110 by means of the heating device 730, by a closed-loop control, for example. Thus, since the temperature can be maintained at a predetermined level by the temperature control unit 700, a reliable measurement result when determining measurement data of the probe fluid 112 can be achieved.

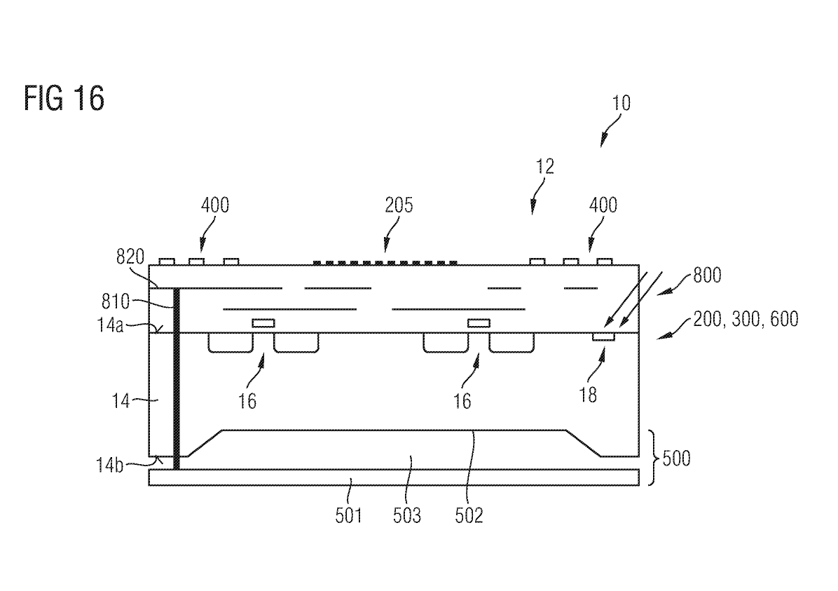

FIG. 16 is a schematic cross-sectional view of a monolithic circuit 12 integrated in a test strip 10 according to an embodiment. As can be seen from FIG. 16, the monolithic circuit 12 comprises a semiconductor body 14 having a first surface 14a and a second surface 14b being opposite to the first surface 14a. On the first surface 14a, a metal wiring layer 800 is provided, which interconnects the electronic components integrated in the semiconductor body 14 such as the sensor unit 200, the communication unit 300 or the energy harvesting unit 600. On the metal wiring layer 800, the antenna unit 400 and the sensor electrode unit 205 may be provided. The integrated circuit of the electronic components such as the sensor unit 200, the communication unit 300 and the energy harvesting unit 600 is indicated by two transistors 16. In addition, an optical component 18 such as a photodiode may be integrated in the semiconductor body 14. Such a photodiode may be employed as an optical sensor 264 as discussed above.

At the second surface 14b of the semiconductor body 14, the energy storage unit 500 may be provided. As can be seen from FIG. 16, the energy storage unit 500 comprises an anode 501, a cathode 502 at the second surface 14b of the semiconductor body 14, and an electrolyte 503 between the anode 501 and the cathode 502 to provide a battery element. The anode 501 is connected to a through-silicon via 810 to electrically connect the anode 501 with the metal wiring layer 800. The cathode 502 is electrically connected to the bulk of the semiconductor body 14. Thus, an on-chip battery is achieved by the structure as shown in FIG. 16 of the monolithic circuit 12 of the test strip 10. The monolithic circuit 12 may further comprise sensors, harvesters, RX/TX circuits, booster antennas, microcontrollers, random access memories, read-only memories, flash memories or clock reference units.

FIG. 17 is a schematic view of a test strip according to an embodiment. As can be seen from FIG. 17, the test strip 10 comprises a printed circuit board (PCB) 15, on which the sensor unit 200, the communication unit 300, and the energy storage unit 500 may be mounted by soldering, for example. An inductive coil antenna 410a may be arranged on the printed circuit board 15 surrounding the sensor unit 200, the communication unit 300, the energy storage unit 500, the energy harvesting unit 600 and the temperature control unit 700 mounted on the printed circuit board 15. However, at least one of the electronic components 500 to 700 may also be omitted. A sensor electrode unit holder 15a may be connected to the printed circuit board 15. On the sensor electrode unit holder 15a, the sensor electrode unit 205 as described above and the fluid reservoir 110 are provided. The sensor electrode unit 205 may be electrically connected with the sensor unit 200 by means of a plug connection.

FIGS. 18A and 18B are schematic views of a test strip 10 according to an embodiment integrated in a cup 60. As can be seen from FIGS. 18A and 18B, the test strip 10 is not restricted to a longitudinal strip form, but may also be adapted to fit into a bottom area of the cup 60. This embodiment may be provided when testing urine of a patient, wherein the cup 60 constitutes the fluid reservoir 110 and the urine of a patient constitutes the probe fluid 112. As can be seen from FIG. 18B, the sensor electrode unit 205, the sensor unit 200, the communication unit 300, the energy storage unit 500, the energy harvesting unit 600 and the temperature control unit 700 are mounted on a printed circuit board 15′. The printed circuit board 15′ has a circle shape. On the printed circuit board 15′, the inductive coil antenna 410a may be arranged surrounding the sensor electrode unit 205, the sensor unit 200, the communication unit 300, the energy storage unit 500, the energy harvesting unit 600, and the temperature control unit 700. However, at least one of the electronic components 500 to 700 may also be omitted.

Thus, according to an embodiment, a test strip 10 comprising an integrated lancet for finger-pricking, an integrated blood-sensor chip, an integrated energy source and a communication module including an antenna for analyzing and transmitting blood-test data to a smart-phone for data display and data registration is provided. Such a test strip 10 allows for a simple and mobile point-of-care-testing by enabling patients to directly use their smart-phone (or tablet/smart watch/laptop/PC) for data display and data registration. The sensor can be an impedance spectrometer or an amperometer or a similar device, which is be able to detect at least one blood-parameter/body-fluid-parameter related to the body condition of patients, like glucose level, infection, hormones, salts, for example.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A test strip, comprising:
a test strip body comprising a fluid reservoir,
a sensor unit configured to determine measurement data of a probe fluid in the fluid reservoir, and
a communication unit electrically connected to the sensor unit, the communication unit including an antenna unit configured to transmit the measurement data, wherein the sensor unit and the communication unit are integrated in a monolithic circuit.

2. The test strip of claim 1, wherein the antenna unit comprises at least one of a radio frequency identification (RFID)/near field communication (NFC) antenna and an radio frequency identification (RFID)/ultra-high frequency (UHF) antenna.

3. The test strip of claim 1, wherein the sensor unit comprises a sensor electrode unit configured to determine amperometric data, voltammetry data, voltage levels or impedance data of the probe fluid.

4. The test strip of claim 3, wherein the antenna unit constitutes the sensor electrode unit.

5. The test strip of claim 1, wherein the sensor unit comprises an optical sensor configured to determine optical data of the probe fluid.

6. The test strip of claim 1, wherein the antenna unit is further configured to receive external configuration data.

7. The test strip of claim 1, further comprising a temperature sensor configured to determine the temperature of the fluid reservoir.

8. The test strip of claim 1, further comprising a temperature control unit configured to regulate the temperature of the fluid reservoir.

9. The test strip of claim 1, further comprising an energy harvesting unit configured to harvest energy from an external power source, the energy harvesting unit being connected to the antenna unit.

10. The test strip of claim 1, further comprising an energy storage unit connected to the sensor unit and the communication unit.

11. The test strip of claim 1, wherein the measurement data is indicative of a glucose concentration, a pH-value, a salt concentration, a potassium concentration, a concentration of a chemical substance, a concentration of a biochemical substance, or a conductivity value of the probe fluid in the fluid reservoir.

12. The test strip of claim 1, further comprising a lancet fixed to the test strip body and configured to penetrate a skin of a test strip user.

13. The test strip of claim 12, wherein the lancet comprises a tube capable to transport the probe fluid by capillary effect to the fluid reservoir.

14. The test strip of claim 12, wherein the test strip body comprises a main part and a folding part, the lancet being fixed to an end portion of the folding part such that the lancet is overlapping the main part in a folded state of the folding part and protruding from the folding part in an unfolded state of the folding part.

15. The test strip of claim 14, further comprising an aseptic package configured to accommodate the test strip body having the folding part in a folded state.

16. The test strip of claim 1, wherein the test strip body comprises a synthetic material.

17. A system for determining measurement data of a probe fluid, comprising a test strip and an external reader,
the test strip comprises a test strip body comprising a fluid reservoir, a sensor unit configured to determine measurement data of the probe fluid in the fluid reservoir, and a communication unit electrically connected to the sensor unit, the communication unit including an antenna unit configured to transmit the measurement data, wherein the sensor unit and the communication unit are integrated in a monolithic circuit, and
the external reader is configured to transmit radio frequency energy powering the test strip and is further configured to receive the measurement data from the test strip.

18. The system of claim 17, wherein the external reader is one of the group comprising a cellular phone, a personal computer, a tablet personal computer, or a watch.
